(19) United States
(12) Patent Application Publication
(10) Pub. No.: US 2026/0212509 A1
(43) Pub. Date: Jul. 23, 2026

(54) PHOTOMETRIC STEREO IMAGE EVALUATION OF CUTTINGS PARTICLES

(71) Applicant: Schlumberger Technology Corporation, Sugar Land, TX (US)

(72) Inventors: Mahdi Ammar, Dhahran (SA); Simone Di Santo, Dhahran (SA)

(21) Appl. No.: 19/034,859

(22) Filed: Jan. 23, 2025

(51) Int. Cl. G06T 7/12; G01N 1/08; G01N 1/34; G01N 33/24; G06T 7/174; G06T 7/187

(52) U.S. Cl. CPC G06T 7/12; G01N 1/08; G01N 1/34; G01N 33/24; G06T 7/174; G06T 7/187; G06T 2207/10012; G06T 2207/30181

(57) ABSTRACT

A method for generating a segmented image of cuttings particles includes acquiring and preparing the cuttings particles for imaging and placing the prepared cuttings particles in front of a digital camera. At least three digital images of the cuttings particles are acquired at corresponding non-coplanar illumination angles and then combined to generate a photometric stereo image of the cuttings particles. The segmented image may be generated from the photometric stereo image.

### CROSS REFERENCE TO RELATED APPLICATIONS

None.

### BACKGROUND

Cuttings particles are produced during drilling operations for oil and gas exploration and recovery, geothermal, and scientific exploration. It will be appreciated that the cuttings particles are abundant in volume and number and may provide one of the lowest cost and most abundant data sources for understanding and characterizing the subsurface rock and formation properties. Cuttings particles have long been evaluated at the surface to generate detailed records of cuttings properties. Moreover, digital images of the cuttings particles are sometimes acquired and later analyzed by offsite geologists. This evaluation is both time consuming and costly.

While the above-described practices for evaluating cuttings particles are commercially serviceable, there is a need for increased automation. In recent years methods have been disclosed for automatically evaluating digital images of cuttings particles using a digital image processing and artificial intelligence (AI) techniques to automatically segment individual cuttings particles, classifying cuttings lithology, and estimate formation porosity. While these new methods are promising, there is room for further improvements.

### BRIEF DESCRIPTION OF THE DRAWINGS

For a more complete understanding of the disclosed subject matter, and advantages thereof, reference is now made to the following descriptions taken in conjunction with the accompanying drawings, in which:

FIG. 1 depicts an example drilling rig including an example system for evaluating cuttings particles.

FIG. 2 depicts a flow chart of an example method for generating a segmented image of drill cuttings particles.

FIG. 3 depicts an example digital image of densely configured cuttings particles disposed on a tray.

FIG. 4 depicts a schematic illustration of a cuttings particle being illuminated from three non-coplanar light directions to acquire corresponding digital images and a photometric stereo image.

FIG. 5 depicts a flow chart of an example method for segmenting individual cuttings particles in an image of densely configured cuttings particles.

FIG. 6 depicts an example implementation in which stationary cuttings particles are illuminated from four distinct light source orientations during image acquisition.

FIG. 7A depicts an example system for generating a segmented image of drill cuttings particles.

FIG. 7B depicts another example system for generating a segmented image of drill cuttings particles.

### DETAILED DESCRIPTION

Embodiments of this disclosure include systems and methods for evaluating cuttings particles generated during a subterranean drilling operation. In one example embodiment, a method for generating a segmented image of cuttings particles comprises acquiring and preparing the cuttings particles for imaging and placing the prepared cuttings particles in front of a digital camera. At least three digital images of the cuttings particles are acquired at corresponding non-coplanar illumination angles and then combined to generate a photometric stereo image of the cuttings particles. A segmented image that identifies individual ones of the cuttings particles may be generated from the photometric stereo image. A characteristic of a subterranean formation may then be estimated from the segmented image.

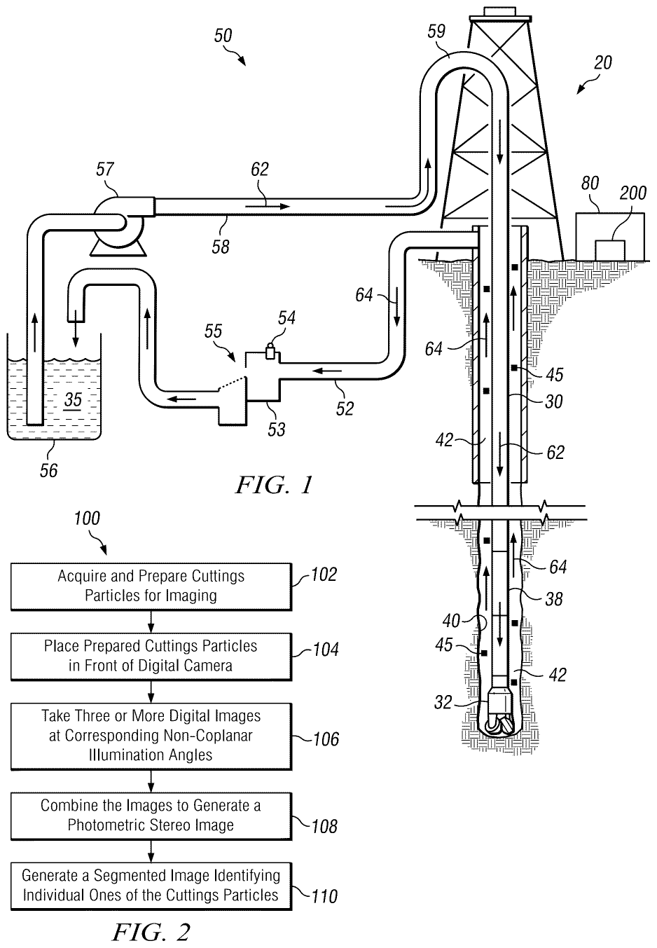

FIG. 1 depicts an example drilling rig 20 including a system 100 for evaluating cuttings particles that are removed from circulating drilling fluid on the rig. The drilling rig 20 may be positioned over a subterranean formation (not shown). The rig 20 may include, for example, a derrick and a hoisting apparatus (also not shown) for raising and lowering a drill string 30, which, as shown, extends into wellbore 40 and includes, for example, a drill bit 32 and one or more downhole measurement tools 38 (e.g., a logging while drilling tool or a measurement while drilling tool) in a bottom hole assembly (BHA) above the bit 32. Suitable drilling systems, for example, including drilling, steering, logging, and other downhole tools are well known in the art.

Drilling rig 20 further includes a surface system 50 for controlling the flow of drilling fluid used on the rig (e.g., used in drilling the wellbore 40). In the example rig depicted, drilling fluid 35 is pumped downhole (as depicted at 62), for example, via a conventional mud pump 57. The drilling fluid 35 may be pumped, for example, through a standpipe 58 and mud hose 59 in route to the drill string 30. The drilling fluid 35 typically emerges from the drill string 30 at or near the drill bit 32 and creates an upward flow 64 of mud through the wellbore annulus 42 (the annular space between the drill string and the wellbore wall). The drilling fluid 35 then flows through a return conduit 52 to a mud pit system 56 where may be recirculated. It will be appreciated that the terms drilling fluid and mud are used synonymously herein.

The circulating drilling fluid 35 is intended to perform many functions during a drilling operation, one of which is to carrying drill cuttings 45 to the surface (in upward flow 64). The drill cuttings 45 are commonly removed from the returning mud via a shale shaker 55 (or other similar solids control equipment) in the return conduit (e.g., immediately upstream of the mud pits 56). Formation gases that are released during drilling may also be carried to the surface in the circulating drilling fluid. These gasses are commonly removed from the fluid, for example, via a degasser or gas trap 54 located in or near a header tank 53 that is immediately upstream of the shale shaker 55 in the example depiction. The drill cuttings 45 may be evaluated to characterize the subterranean formation and/or estimate various properties thereof as described in more detail below.

The rig 20 may include a system 200 configured to take and evaluate digital images of the drill cuttings as described in more detail below. The system 200 may be deployed at the rig site (e.g., in an onsite laboratory 80) or offsite. However, the disclosed embodiments are not limited in this regard. The system 200 may include computer hardware and software configured to automatically or semi-automatically evaluate the cuttings images. To perform these functions, the hardware may include one or more processors (e.g., microprocessors) which may be connected to one or more data storage devices (e.g., hard drives or solid state memory). As is known to those of ordinary skill, the processors may be further connected to a network, e.g., to receive the images from a networked camera system (not shown) or another computer system. It will, of course, be understood that the disclosed embodiments are not limited the use of or the configuration of any particular computer hardware and/or software.

While FIG. 1 depicts a land rig 20, it will be appreciated that the disclosed embodiments are equally well suited for land rigs or offshore rigs. As is known to those of ordinary skill, offshore rigs commonly include a platform deployed atop a riser that extends from the sea floor to the surface. The drill string extends downward from the platform, through the riser, and into the wellbore through a blowout preventer (BOP) located on the sea floor. The disclosed embodiments are not limited in these regards.

In recent years methods have been disclosed for automatically evaluating digital images of cuttings particles using digital image processing and artificial intelligence (AI) techniques to automatically segment individual cuttings particles and classifying cuttings lithology. While these new methods are promising, there are inherent difficulties with image segmentation of overlapping or piled objects, such as small rock cuttings, owing to the limitations of 2D imaging, which fails to fully capture the 3D information of the objects.

For example, cuttings particles have complex and irregular shapes, leading to occlusion where one object can hide or partially cover another. In a 2D image, it is challenging to distinguish between individual objects that are overlapping or touching, making accurate segmentation difficult. Moreover, in a 2D image the information about the depth or height of objects is lost making it difficult to determine which parts of the particles are closer to the camera and which are farther away. This can lead to ambiguities in segmentation. Shadowing can add additional complexities to the segmentation task since distinguishing between actual object boundaries and shadow edges can be difficult in 2D. Moreover, cuttings particles often have similar textures or color patterns, making it challenging for traditional 2D image processing techniques to differentiate between them accurately. Still further, the arrangement of cuttings particles on a tray or in a pile of particles may vary significantly with different shapes, densities, and particle orientations, making it difficult to create a one-size-fits-all segmentation solution.

A further difficulty is that complex segmentation tasks require training deep learning algorithms using large data sets of annotated images. Data annotation for deep learning segmentation incurs labor expenses for manual labeling, tool and infrastructure costs, and additional efforts for quality control. Data training costs involve investing in powerful hardware, longer training times, and fine-tuning hyperparameters. Striking a balance between these expenses and the potential benefits is crucial to ensure a cost-effective and successful deployment of a deep learning solution.

Moreover, retraining the deep learning algorithms on specific use cases is often required to achieve optimal results. Such retraining is a complex and time-consuming process that requires access to relevant data, expertise in model tuning, and an understanding of the specific use case difficulties. For effective retraining, sufficient and diverse data representing the specific use case is required. Acquiring and preparing such data may be challenging or even prohibitive if the use of the data is restricted to specific project or geography. Retraining can also be computationally intensive, necessitating powerful hardware and adequate time for training. After retraining, thorough validation is essential to ensure that the model's performance has indeed improved on the target case without adversely affecting performance on other scenarios. This validation process may also be time-consuming and resource intensive. While retraining may (and often does) lead to improved results for a specific case, it may adversely impact the model's performance on other tasks or scenarios. For these and other reasons there is a need in the industry for improved methods for cuttings particle image acquisition and deep learning segmentation methods.

FIG. 2 depicts a flow chart of an example method 100 for generating a segmented image of cuttings particles (e.g., piled cuttings particles). Cuttings particles are acquired and prepared for imaging at 102. The cuttings particles are placed in front of a digital camera at 104. In example embodiments, the cuttings particles may be touching one another. In still other example embodiments at least a portion of the cuttings particles may be piled atop other ones of the cuttings particles. At least three (e.g., at least 4) digital images of the cuttings particles are acquired at corresponding non-coplanar illumination angles at 106. The acquired digital images are combined at 108 to generate a stereo image. Individual particles in the piled cuttings particles are identified in the stereo image at 110 to generate the segmented image. The segmented image may be optionally be further processed to estimate one or more formation characteristics such as a formation lithology and/or a formation porosity as disclosed in commonly assigned US Patent Publication 2023/0220770 and WIPO Publication WO 2024/020523.

The disclosed segmenting methodology may have several advantages over deep learning or artificial intelligence methods for object segmentation. For example, the disclosed embodiments may provide depth information, thereby leading to better separation of particles in the image and may further enable accurate three dimensional (3D) reconstruction. Moreover, such 3D reconstruction may further enhance particle to particle and within particle texture variations. Furthermore, the disclosed embodiments do not rely heavily on the use of annotated data for training and thereby may significantly reduce expense and time. In example embodiments real-time segmentation may be achieved with minimal computation time.

The cuttings particles may be acquired and prepared at 102 of method 100, for example, by drilling wellbore into or through a subterranean formation of interest, for example, using the example rig 20 described above with respect to FIG. 1. The cuttings particles generated while drilling are transported to the surface in the upwardly flowing drilling fluid (e.g., as depicted in FIG. 1). The cuttings particles may be collected, for example, using a shale shaker or other solids separation/control equipment on the rig floor. The collected particles are generally contaminated with oil-based mud (OBM) or water-based mud (WBM) such that further preparation may be required.

To remove such contamination, the cuttings particles may be cleaned at, for example, in a cleaning solution including a suitable solvent (e.g., acetone, ethanol, isopropyl alcohol, or water) and a detergent or surfactant. The cleaning solution is intended to effectively dissolve or soften drilling fluid or other contaminants that are adhered to the particle surfaces. The cleaning may further include physical abrasion or agitation to promote contaminant removal. Such processes may include, for example, mechanical brushing or scraping, liquid jets, compressed air, and/or ultrasonic agitation. After cleaning the cuttings particles may be rinsed with clean water to remove any residual cleaning solution, detergent, and/or solvent. The particles may then be dried and placed on a tray for imaging.

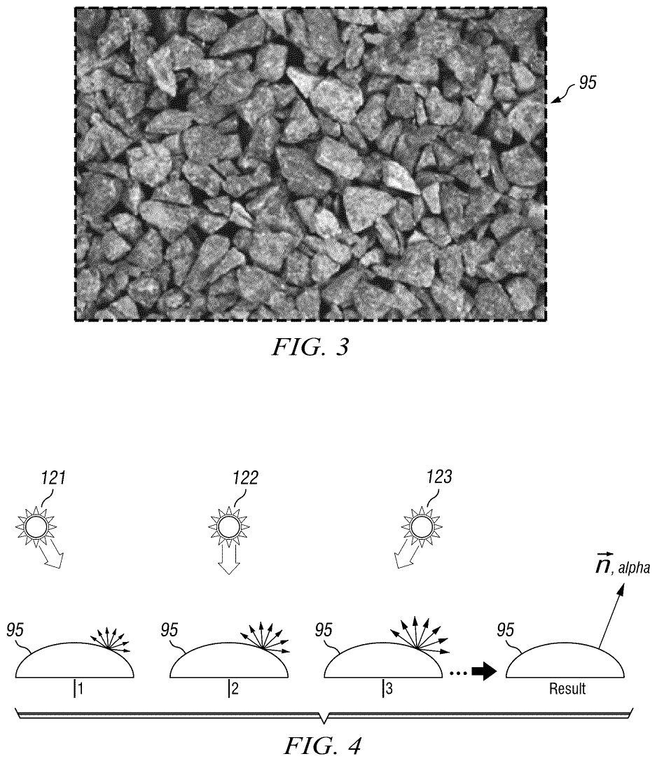

FIG. 3 depicts an example digital image of cuttings particles 95 disposed on a tray (e.g., as placed in front of the digital camera at 104 in FIG. 2). Note that the cuttings particles are in a dense configuration in which individual particles contact one another, sometimes partially overlap other particles, and may even be piled onto each other. This is in contrast to a sparse configuration in which none of the particles touch each other or overlap (no such configuration is shown). It will be appreciated that the disclosed embodiments may be advantageously utilized to evaluate digital images of dense particle configurations, such as shown on FIG. 3.

It will be appreciated that one of the primary challenges to imaging dense configurations of cuttings particles is producing images with ample contrast to effectively distinguish individual particles from one another while maintaining within particle contrast for evaluating texture and textural differences. As depicted in FIG. 3, the individual particles may exhibit variations in elevation, such as rising from or recessing into the image plane as well as possessing their own unique textures and colors. There is a need to improve and enhance image contrast for particle segmentation and subsequent characterization, particularly for densely packed particles.

While the disclosed embodiments may be advantageously utilized to generate segmented images of piled cuttings particles, it will be appreciated that the disclosure is not so limited. Moreover, it will be appreciated that in more sparse configurations it may be advantageous to place the cuttings particles on a tray having a high contrast (vivid) background color to enhance subsequent particle identification and segmentation in the acquired images. Example colors include pure magenta (e.g., with RGB values of 255, 0, 255), pure blue (e.g., with RGB values of 0, 0, 255), pure green (e.g., with RGB values of 0, 255, 0), and so forth. In general, such colors do not exist in nature and, accordingly, may enhance the disclosed segmentation methods, however the disclosed embodiments are expressly not limited in this regard.

Turning now to FIG. 4, stereo image generation at 106 and 108 is described in more detail. Photometric stereo combines images obtained with varying directional illumination to analyze shadows and reflections and thereby enhance image contrast. In the FIG. 4 schematic illustration, cuttings particle 95 is illuminated using at least three non-coplanar light directions 121, 122, and 123 to acquire at least three corresponding digital images. Shading and shadows on the object's surface change with varying light conditions, making it difficult to capture all surface details in a single image. By observing the object under different lighting conditions from the same viewpoint, photometric stereo analyzes variations in intensity to assess (compute) surface normal vectors n and albedo reflectivity (alpha) at selected pixels in the image (e.g., at each of the pixels).

The analysis assumes a fixed camera position and constant camera settings during image capture (the only change being the illumination direction as shown). The resulting images are combined to create a composite image, allowing for local estimates of surface orientation and curvature. The analysis may be based on Lambertian reflectance, which assumes ideal matte surfaces with uniform radiation in all directions, but may be further expanded to accommodate non-Lambertian reflectance models such as Phong, Torrance-Sparrow, and Ward models, broaden the technique's potential.

For example, the photometric stereo operations may utilize material reflectance properties and object surface curvature to calculate an enhanced image based on Lambertian (matte, diffuse) surfaces. The diffuse reflected intensity (I) is proportional to the angle between the incident light direction (L) and the surface normal (n) of the object, driven by the albedo reflectivity (alpha) following Lambert's Cosine Law. The albedo represents the fraction of incident sunlight that the surface reflects. The surface normal (n) and the albedo reflectivity (alpha) may be determined from images acquired from at least three non-coplanar light directions and assuming distant light and parallel rays, the known light directions (L) are predetermined within the illumination setup where (I=alpha * \vec{L}·\vec{n}).

The computed surface normal vectors may advantageously reveal essential information about the cuttings particles surface(s), even uncovering surface irregularities such as scratches, chips, indentations, and/or etch patters despite the expectation of a smooth surface. Moreover, the enhanced contrast may enable improved particle segmentation. While a minimum of three images are generally required to determine the normal, practical applications may use more images (such as four or more or even five or more) to reduce inherent imaging noise and improve image accuracy. The redundancy from multiple images may yield better analysis results, typically necessitating a minimum of four images.

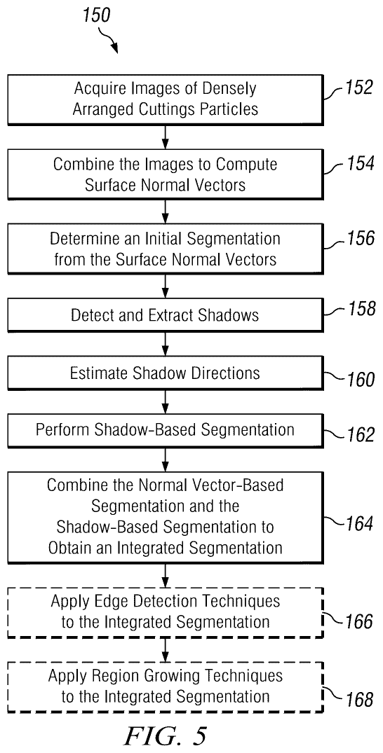

Turning now to FIG. 5, a flow chart of an example method 150 for segmenting individual cuttings particles in a dense configuration (e.g., at 110 of method 100 in FIG. 2) is shown. Method 150 advantageously integrates (or combines) surface normal and shadow information to achieve a more robust segmentation of dense particle configurations. The disclosed embodiments advantageously leverage both geometric and shading cues to achieve more accurate and robust object segmentation and may be particularly advantageous when the cuttings particles have complex shapes and textures and/or are densely configured (e.g., piled upon one another).

With continued reference to FIG. 5, at least three (e.g., at least 4) digital images of the cuttings particles are acquired at corresponding non-coplanar illumination angles at 152 (e.g., as described above with respect to FIG. 2). The acquired digital images are combined at 154 to compute the surface normal vectors. An initial segmentation may be determined at 156 based on the computed surface normal vectors. Shadows are detected and extracted from the image at 158. Such shadow extraction may make use of any suitable algorithm such as thresholding, gradient-based methods, or machine learning. Shadow directions are estimated at 160, for example, via analyzing shadow lengths and orientations in relation to the light source directions. Shadow-based segmentation may be performed at 162, for example, utilizing the shadow information obtained at 160 and thereby distinguish individual particles from one another. The shadows made act as or provide additional boundaries between particles and/or the background between different particles. By considering shadow cues, separate particles may be identified more accurately. The surface normal segmentation obtained at 156 and the shadow-based segmentation obtained at 162 may be combined or integrated at 164 to obtain an improved segmentation. Optional edge detection techniques may be applied to the integrated segmentation at 166 to further refine particle boundaries. Moreover, optional region growing techniques may be applied to the integrated segmentation at 168 to obtain an improved segmentation. For example, pixels having similar normal vectors and shading properties may be grouped together to segment meaningful particle regions. Still further, postprocessing techniques such as morphological operations, noise reduction, and object emerging/splitting may be optionally applied to improve the final segmentation results.

It will be appreciated that the assumption of illumination with distant light having parallel illumination rays is reasonable in many imaging applications, for example, when the dimensions of the illumination system are selected for the scene. Various appropriate lighting tools, such as segment bars and ring lights offered by companies like Advanced Illumination (Rochester, VT), CCS (Boston, MA), or Smart Vision Lights (Muskegon, MI), are readily available and may be utilized. Such purpose-built lights greatly facilitate integration and setup, particularly for machine vision software providers like Matrox Imaging (Montreal, QC, Canada) offering photometric stereo tools.

It will be further appreciated that when the lighting directions and intensities are known, photometric stereo can be effectively solved as a linear system. The lighting positions may be determined based on the illumination setup geometry or calibrated from images using a specular reflective sphere. However, when the illumination details are unknown, a more challenging problem arises, namely uncalibrated photometric stereo. While solutions exist for calculating photometric stereo under these circumstances, it should be noted that uncalibrated methods are more sensitive to acquisition conditions and may suffer from poor repeatability, highlighting the importance of having accurate lighting information for reliable results.

In certain example embodiments, the cuttings particles remain stationary during image acquisition and estimated surface normal vectors and albedo (alpha) results are computed. The albedo result may provide an estimated percentage of the reflected intensity of the cuttings particle surface(s), revealing changes in surface reflectance from shiny to dull. These variations in diffuse reflectivity may indicate differences in material properties, resulting in enhanced visual contrast. Such contrast enhancements are beneficial for segmentation and subsequent image analysis. Sharp changes in surface normal may indicate the presence of defects such as cracks, scratches, or dents. Besides identifying defects, surface normal vectors may be further processed to estimate local surface curvatures. Analyzing these curvature results may be more intuitive than the entire field of normal vectors, as they emphasize local variations in normal directions, such as protruding marks or depressions on an otherwise flat surface.

In other example embodiments, the cuttings particles may be moving, for example, on a conveyor during image acquisition. To accommodate object motion, additional leading and trailing images may be taken with full illumination. These additional images may be used to determine the object's displacement, allowing for realignment of the images to compensate for the object's position at different times. The images between the leading and trailing images (the photometric stereo source images captured with directional lighting) are then translated to ensure that the object's position is consistent in each image. High-speed cameras may be advantageously used to minimize perspective distortion and parallax errors caused by the small degree of displacement during object motion.

In some embodiments, using four distinct lights may be challenging in certain setups or for some objects due to physical constraints or practical limitations. One possible alternative to using multiple fixed lights is to rotate the sample (the tray of cuttings particles) using a rotating stage. This technique involves rotating the tray of cuttings particles instead of positioning multiple fixed lights around it. For example, a single light source positioned at a fixed angle relative to the object may be used to illuminate the object from a consistent direction throughout the rotation. The particles may be placed on a turntable (or other rotational device) that allows it to be rotated smoothly and accurately around a single axis. Images may then be captured at predetermined angular orientations during rotation (e.g., at 60 or 90 degree intervals). The number of images taken may depend on the level of accuracy and detail required for the reconstruction with more images providing better 3D shape reconstruction at the expense of increased time and processing requirements.

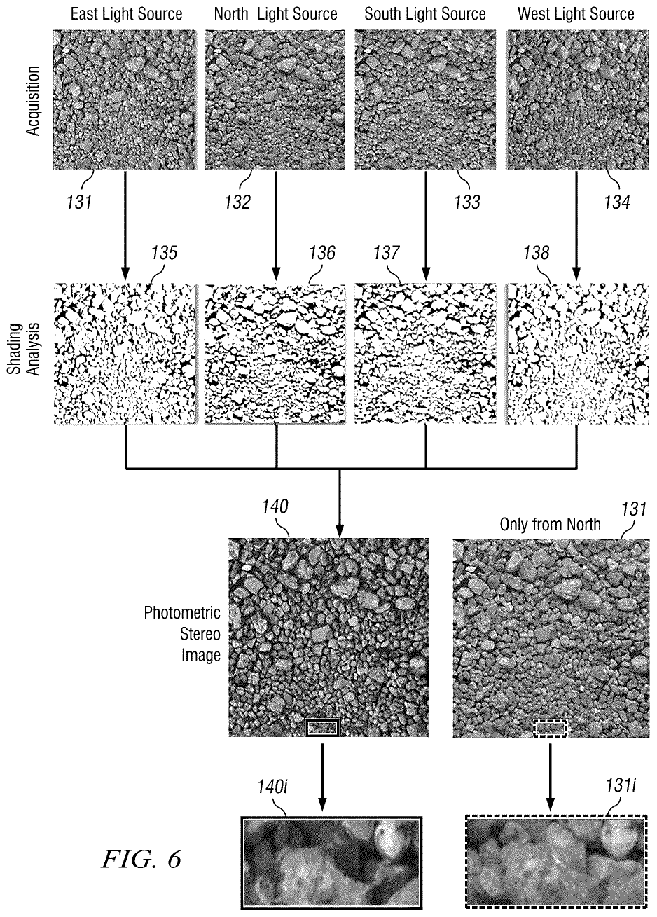

FIG. 6 depicts an example implementation in which the cuttings particles remain stationary during image acquisition. In this example, four distinct images 131, 132, 133, and 134 of piled cutting particles are generated at corresponding distinct light source orientations (referred to herein as East, North, South, and West). Note that the cuttings particles are piled and densely configured as described above with respect to FIG. 3. Moreover, the particles have a large range of sizes and shapes. FIG. 6 further depicts a shading analysis of each image at 135, 136, 137, and 138. A final photometric stereo image is depicted at 140. As depicted, the final photometric stereo image 140 has shaper contrast and boundary delineation than any of the individual images (shown side by side with image 131). Such contrast and boundary delineation may be particularly well observed at 140i and 131i. Such contrast and particle delineation may advantageously improve the efficiency and accuracy of particle segmentation algorithms, for example, as described above.

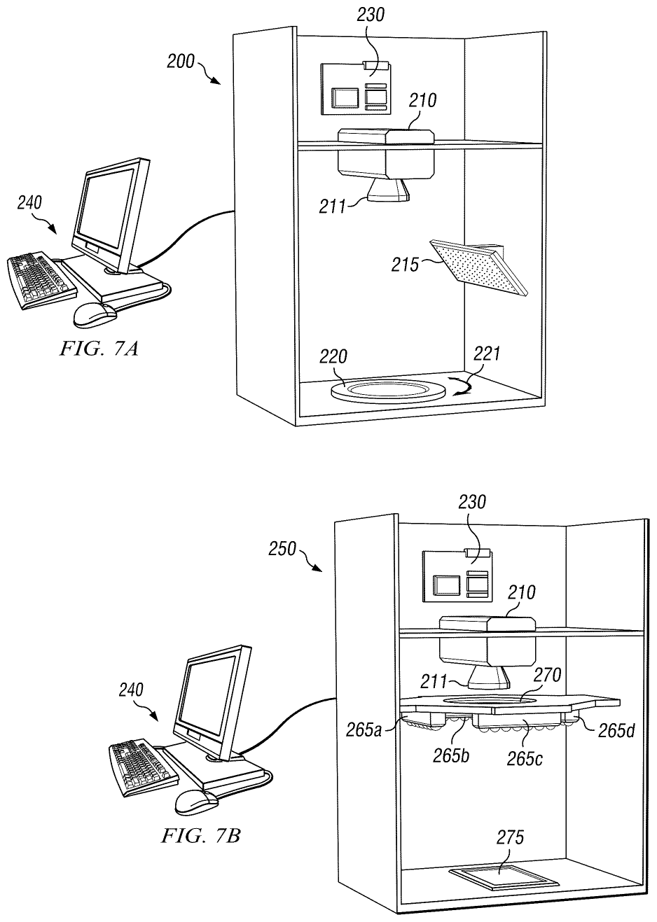

FIG. 7 depicts an example system 200 for generating a segmented image of drill cuttings particles. It will be appreciated that the disclosed embodiments are not limited to any particular system configuration. As described above, the example system 200 may include a stage or tray 220 configured to receive the cuttings particles. The tray may be sized and shaped (in coordination with a camera lens system) to hold a sufficient quantity of cuttings and to fill a field of view in an image acquisition device. The system 200 may include substantially any suitable tray, for example, including a plastic or metal tray as described above, however in preferred embodiments includes a rotatable stage as described above and indicated at 221.

The system 200 may further include at least one light source 215, for example, including a white light source configured to illuminate the sample holder 220 (and cuttings placed on the tray). The light source 215 may include, for example, a light emitting diode (LED, diode array, or other suitable light sources capable of illuminating cuttings particles on the holder 220. The system 200 may further include a camera 210 and a corresponding lens 211 deployed above the sample holder 220, for example, mounted on or in a divider disposed between upper and lower chambers of the system 220. The camera 210 may advantageously include a high resolution color camera, for example, including a 10 or 20 (or more) megapixel image sensor. Substantially any suitable lens 211 may be utilized. The lens 211 may be configured to provide sharp (focused) images to the image sensor, for example, including a 25 mm lens. The lens 211 may alternatively include a variable zoom lens.

The system 200 may further include a controller 230 such as a computer board (or motherboard) configured to control operation of the camera 210, the lens 211, lights 215, and/or rotating sample holder 220. The controller 200 may include one or more processors (e.g., microprocessors) which may be connected to one or more data storage devices (e.g., hard drives or solid state memory). The controller may be configured to network (e.g., communicate) with external devices (e.g., an external computer system 240), for example, via a hard wire or wireless connection. For example, the controller 230 may be configured to upload acquired images to the computer system 240 for processing as described in FIGS. 2 and 5. In such embodiments, the controller 230 and/or computer system 240 may include processor executable instructions stored in memory to execute selected ones of the method steps described above with respect to FIGS. 2 and 5.

FIG. 7B another example system 250 for generating a segmented image of drill cuttings particles. System 250 is similar to system 200 in that it includes a stage or tray 275 configured to receive the cuttings particles and a camera 210 and a corresponding lens 211 deployed above the sample holder 275 and configured to acquire digital images of the cuttings particles in the tray. System 250 may further include a controller 230 and computer system 240 as described above with respect to FIG. 7A. In FIG. 7B, the system 250 includes first, second, third, and fourth lights 265a, 265b, 265c, 265d (e.g., bar lights) deployed about and configured to illuminate the sample holder (e.g., from four distinct directions spaced at angular intervals of about 90 degrees). Such a system 250 may be advantageously utilized with or without a rotatable tray or stage (a nonrotating stage 275 is depicted). In certain advantageous embodiments, the lights 265a, 265b, 265c, 265d may be deployed on a platform 270 (or divider) and directed to illuminate cuttings particles on the nonrotating stage 275. The lights 265a, 265b, 265c, 265d may alternatively be deployed on the sidewalls of the chamber. The disclosed embodiments are of course not limited in this regard. Moreover, it will be appreciated that the disclosed embodiments are not limited to the use of a rotatable stage (as depicted on FIG. 7A) or the use of multiple lights (as depicted on FIG. 7B) so long as the system is capable of acquiring three or more images a corresponding illumination directions.

With continued reference to FIGS. 7A and 7B, it will be appreciated that systems 200 and 250 may be utilized to acquire the necessary plurality of digital images and in turn process or evaluate those images to obtain the segmented image. For example, in FIG. 7A, system 200 may be employed to rotate the rotatable stage 220 to at least three distinct angular orientations (e.g., to four distinct angular orientations) and acquire a distinct digital image of the cuttings particles at each corresponding angular orientations. In FIG. 7B, system 250 may be employed to selectively and individually illuminate each of the light sources (e.g., lights 265a, 265b, 265c, 265d) and acquire a distinct digital image of the cuttings particles corresponding to each of the individual illuminations.

It will be understood that the present disclosure includes numerous embodiments. These embodiments include, but are not limited to, the following embodiments.

In a first embodiment, a method for generating a segmented image of cuttings particles, comprises acquiring and preparing the cuttings particles for imaging; placing the prepared drill cuttings particles in front of a digital camera; acquiring at least three digital images of the cuttings particles at corresponding non-coplanar illumination angles; combining the at least three digital images to generate a photometric stereo image of the cuttings particles; and generating a segmented image identifying individual ones of the cuttings particles.

A second embodiment may include the first embodiment, wherein the acquiring and preparing further comprises drilling a subterranean wellbore; collecting the cuttings particles from circulating drilling fluid; washing the collected cuttings particles; and rinsing the washed cuttings particles.

A third embodiment may include any one of the first through second embodiments, wherein the placing comprises placing the prepared drill cuttings particles on a rotatable stage in front of the digital camera; and the acquiring comprises rotating the rotatable stage to at least three distinct angular orientations and acquiring a digital image of the cuttings particles at each of the at least three distinct angular orientations.

A fourth embodiment may include any one of the first through third embodiments, wherein the placing comprises placing the prepared drill cuttings particles on a stage in front of the digital camera and at least three light sources configured to illuminate the stage at correspondingly distinct angular orientations; and the acquiring comprises selectively and individually illuminating each of the at least three light sources and acquiring a digital image of the cuttings particles corresponding to each of the individual illuminations of the at least three light sources.

A fifth embodiment may include any one of the first through fourth embodiments, wherein the combining comprises computing surface normal vectors n and albedo reflectivity (alpha) at selected pixels in the photometric stereo image; and the generating the segmented image further comprises identifying individual ones of the cuttings particles based on the computed surface normal vectors.

A sixth embodiment may include any one of the first through fifth embodiments, wherein the generating the segmented image further comprises detecting and extracting shadows from the at least three digital images; estimating directions of selected ones of the extracted shadows; and identifying individual ones of the cuttings particles based on the estimated shadow directions.

A seventh embodiment may include any one of the first through sixth embodiments, wherein the generating the segmented image further comprises generating a first segmented image in which individual ones of the cuttings particles are identified based on computed surface normal vectors in the photometric stereo image; and generating a second segmented image in which individual ones of the cuttings particles are identified from estimated directions of shadows extracted from the at least three digital images.

An eighth embodiment may include the seventh embodiment, wherein the generating the segmented image further comprises combining the first segmented image and the second segmented image to obtain a third segmented image.

A ninth embodiment may include the eighth embodiment, wherein the generating the segmented image further comprises applying edge detection techniques or region growing techniques to the third segmented image.

A tenth embodiment may include any one of the first through ninth embodiments, wherein the acquiring, the combining, and the generating are performed automatically.

An eleventh embodiment may include any one of the first through tenth embodiments, further comprising estimating a characteristic of a subterranean formation from the segmented image.

In a twelfth embodiment, a system for generating a segmented image of cuttings particles comprises a sample holder configured to receive cuttings particles; a digital camera positioned and configured to record digital images of the cuttings particles on the sample holder; at least one light source configured to illuminate the sample holder; and a controller configured to (i) cause the digital camera to acquire at least three digital images of the cuttings particles at corresponding non-coplanar illumination angles; (ii) combine the at least three digital images to generate a photometric stereo image of the cuttings particles; and (iii) generate a segmented image identifying individual ones of the cuttings particles.

A thirteenth embodiment may include the twelfth embodiment, wherein the controller is further configured to rotate the sample holder to at least three distinct angular orientations corresponding to the non-coplanar illumination angles when acquiring the at least three digital images of the cuttings particles.

A fourteenth embodiment may include any one of the twelfth through thirteenth embodiments, wherein the combine the at least three digital images further comprises compute surface normal vectors n and albedo reflectivity (alpha) at selected pixels in the photometric stereo image.

A fifteenth embodiments may include the fourteenth embodiment, wherein the generate the segmented image further comprises generate a first segmented image in which individual ones of the cuttings particles are identified based on the computed surface normal vectors in the photometric stereo image; generate a second segmented image in which individual ones of the cuttings particles are identified from estimated directions of shadows extracted from the at least three digital images; and combine the first segmented image and the second segmented image to obtain a third segmented image.

In a sixteenth embodiment, a method for generating a segmented image of cuttings particles comprises acquiring and preparing the cuttings particles for imaging; placing the prepared drill cuttings particles in front of a digital camera; acquiring at least three digital images of the cuttings particles at corresponding non-coplanar illumination angles; combining the at least three digital images to generate a photometric stereo image of the cuttings particles; generating a first segmented image in which individual ones of the cuttings particles are identified based on computed surface normal vectors in the photometric stereo image; and generating a second segmented image in which individual ones of the cuttings particles are identified from estimated directions of shadows extracted from the at least three digital images; and combining the first segmented image and the second segmented image to obtain a third segmented image.

A seventeenth embodiment may include the sixteenth embodiment, wherein the acquiring and preparing further comprises drilling a subterranean wellbore; collecting the cuttings particles from circulating drilling fluid; washing the collected cuttings particles; and rinsing the washed cuttings particles.

An eighteenth embodiment may include any one of the sixteenth through seventeenth embodiments, wherein the placing comprises placing the prepared drill cuttings particles on a rotatable stage in front of the digital camera; and the acquiring comprises rotating the rotatable stage to at least three distinct angular orientations and acquiring a digital image of the cuttings particles at each of the at least three distinct angular orientations.

A nineteenth embodiment may include any one of the sixteenth through eighteenth embodiments, wherein the placing comprises placing the prepared drill cuttings particles on a stage in front of the digital camera and at least three light sources configured to illuminate the stage at correspondingly distinct angular orientations; and the acquiring comprises selectively and individually illuminating each of the at least three light sources and acquiring a digital image of the cuttings particles corresponding to each of the individual illuminations of the at least three light sources.

A twentieth embodiment may include any one of the sixteenth through nineteenth embodiments, further comprising applying edge detection techniques or region growing techniques to the third segmented image.

A twenty-first embodiment may include any one of the sixteenth through twentieth embodiments, further comprising estimating a characteristic of a subterranean formation from the third segmented image.

Although photometric stereo image of cuttings particles has been described in detail, it should be understood that various changes, substitutions and alternations can be made herein without departing from the spirit and scope of the disclosure as defined by the appended claims.

1. A method for generating a segmented image of cuttings particles, the method comprising:
acquiring and preparing the cuttings particles for imaging;
placing the prepared cuttings particles in front of a digital camera;
acquiring at least three digital images of the cuttings particles at corresponding non-coplanar illumination angles;
combining the at least three digital images to generate a photometric stereo image of the cuttings particles; and
generating a segmented image identifying individual ones of the cuttings particles.

2. The method of claim 1, wherein the acquiring and preparing further comprises:
drilling a subterranean wellbore;
collecting the cuttings particles from circulating drilling fluid;
washing the collected cuttings particles; and
rinsing the washed cuttings particles.

3. The method of claim 1, wherein:
the placing comprises placing the prepared drill cuttings particles on a rotatable stage in front of the digital camera; and
the acquiring comprises rotating the rotatable stage to at least three distinct angular orientations and acquiring a digital image of the cuttings particles at each of the at least three distinct angular orientations.

4. The method of claim 1, wherein:
the placing comprises placing the prepared drill cuttings particles on a stage in front of the digital camera and at least three light sources configured to illuminate the stage at correspondingly distinct angular orientations; and
the acquiring comprises selectively and individually illuminating each of the at least three light sources and acquiring a digital image of the cuttings particles corresponding to each of the individual illuminations of the at least three light sources.

5. The method of claim 1, wherein:
the combining comprises computing surface normal vectors n and albedo reflectivity at selected pixels in the photometric stereo image; and
the generating the segmented image further comprises identifying individual ones of the cuttings particles based on the computed surface normal vectors.

6. The method of claim 1, wherein the generating the segmented image further comprises:
detecting and extracting shadows from the at least three digital images;
estimating directions of selected ones of the extracted shadows; and
identifying individual ones of the cuttings particles based on the estimated shadow directions.

7. The method of claim 1, wherein the generating the segmented image further comprises:
generating a first segmented image in which individual ones of the cuttings particles are identified based on computed surface normal vectors in the photometric stereo image; and
generating a second segmented image in which individual ones of the cuttings particles are identified from estimated directions of shadows extracted from the at least three digital images.

8. The method of claim 7, wherein the generating the segmented image further comprises combining the first segmented image and the second segmented image to obtain a third segmented image.

9. The method of claim 8, wherein the generating the segmented image further comprises applying edge detection techniques or region growing techniques to the third segmented image.

10. The method of claim 1, wherein the acquiring, the combining, and the generating are performed automatically.

11. The method of claim 1, further comprising estimating a characteristic of a subterranean formation from the segmented image.

12. A system for generating a segmented image of cuttings particles, the system comprising:
a sample holder configured to receive cuttings particles;
a digital camera positioned and configured to record digital images of the cuttings particles on the sample holder;
a light source configured to illuminate the sample holder; and
a controller configured to (i) cause the digital camera to acquire at least three digital images of the cuttings particles at corresponding non-coplanar illumination angles; (ii) combine the at least three digital images to generate a photometric stereo image of the cuttings particles; and (iii) generate a segmented image identifying individual ones of the cuttings particles.

13. The system of claim 12, wherein the controller is further configured to rotate the sample holder to at least three distinct angular orientations corresponding to the non-coplanar illumination angles when acquiring the at least three digital images of the cuttings particles.

14. The system of claim 12, wherein the combine the at least three digital images further comprises compute surface normal vectors n and albedo reflectivity (alpha) at selected pixels in the photometric stereo image.

15. The system of claim 14, wherein the generate the segmented image further comprises:
generate a first segmented image in which individual ones of the cuttings particles are identified based on the computed surface normal vectors in the photometric stereo image;
generate a second segmented image in which individual ones of the cuttings particles are identified from estimated directions of shadows extracted from the at least three digital images; and
combine the first segmented image and the second segmented image to obtain a third segmented image.

16. A method for generating a segmented image of cuttings particles, the method comprising:
acquiring and preparing the cuttings particles for imaging;
placing the prepared drill cuttings particles in front of a digital camera;
acquiring at least three digital images of the cuttings particles at corresponding non-coplanar illumination angles;
combining the at least three digital images to generate a photometric stereo image of the cuttings particles;
generating a first segmented image in which individual ones of the cuttings particles are identified based on computed surface normal vectors in the photometric stereo image; and
generating a second segmented image in which individual ones of the cuttings particles are identified from estimated directions of shadows extracted from the at least three digital images; and
combining the first segmented image and the second segmented image to obtain a third segmented image.

17. The method of claim 16, wherein the acquiring and preparing further comprises:
drilling a subterranean wellbore;
collecting the cuttings particles from circulating drilling fluid;
washing the collected cuttings particles; and
rinsing the washed cuttings particles.

18. The method of claim 16, wherein:
the placing comprises placing the prepared drill cuttings particles on a rotatable stage in front of the digital camera; and
the acquiring comprises rotating the rotatable stage to at least three distinct angular orientations and acquiring a digital image of the cuttings particles at each of the at least three distinct angular orientations.

19. The method of claim 16, further comprising applying edge detection techniques or region growing techniques to the third segmented image.

20. The method of claim 16, further comprising estimating a characteristic of a subterranean formation from the third segmented image.
